(19) United States
(12) Patent Application Publication
(10) Pub. No.: US 2025/0249003 A1
(43) Pub. Date: Aug. 7, 2025

(54) COMBINATIONS OF KRAS G12D INHIBITORS WITH IRINOTECAN AND RELATED METHODS OF TREATMENT

(71) Applicant: MIRATI THERAPEUTICS, INC., San Diego, CA (US)

(72) Inventors: Jill Hallin, San Diego, CA (US); James Gail Christensen, San Diego, CA (US); Vickie Bowcut, San Diego, CA (US); Peter Olson, San Diego, CA (US)

(21) Appl. No.: 18/695,032

(22) PCT Filed: Oct. 4, 2022
(86) PCT No.: PCT/US2022/045625

§ 371 Date: Mar. 25, 2024

Related U.S. Application Data
(60) Provisional application No. 63/252,405, filed on Oct. 5, 2021.

(51) Int. Cl. A61K 31/519; A61K 31/475; A61P 35/00

(52) U.S. Cl. CPC A61K 31/519; A61K 31/475; A61P 35/00

(57) ABSTRACT

The present invention relates to combination therapies for treating KRas G12D cancers. In particular, the present invention relates to methods of treating cancer in a subject in need thereof, comprising administering to the subject a therapeutically effective amount of a combination of irinotecan (or an analog thereof) and a KRas G12D inhibitor, pharmaceutical compositions comprising a such compositions, kits comprising such compositions and methods of use thereof.

### FIELD OF THE INVENTION

The present invention relates to combination therapies useful for treating cancer. In particular, the present invention relates to therapeutically effective combinations of a KRas G12D inhibitor and irinotecan, and additionally pharmaceutical compositions comprising theses agents, kits comprising such compositions, and methods of use thereof.

### BACKGROUND OF THE INVENTION

### KRas Inhibitors

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79 (11): 3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13 (12): 928-942 doi: 10.1038/nrd428). Single nucleotide substitutions that result in missense mutations at codons 12 and 13 of the KRas primary amino acid sequence comprise approximately 33% of these KRas driver mutations in lung adenocarcinoma, with a G12D mutation being a common activating mutation (e.g., see Li, Balmain and Counter, (2018) Nat Rev Cancer Dec; 18 (12): 767-777; Sanchez-Vega, et al, (2018) Cell; 173, 321-337).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, only a single KRas G12C inhibitor (the KRas G12C inhibitor sotorasib) has demonstrated sufficient safety and/or efficacy to obtain regulatory approval (e.g., see: FDA Approves First KRAS Inhibitor: Sotorasib. [No authors listed] Cancer Discov. 2021 August; 11 (8): OF4. doi: 10.1158/2159-8290. CD-NB2021-0362. Epub 2021 Jun. 22). To date, no KRas G12D inhibitors have demonstrated sufficient safety and/or efficacy to obtain regulatory approval.

Compounds that inhibit KRas activity are still highly desirable and under investigation, including those that disrupt effectors such as guanine nucleotide exchange factors (e.g., see Sun et al., (2012) Agnew Chem Int Ed Engl. 51 (25): 6140-6143 doi: 10.1002/anie201201358) as well as those that target KRas G12D (e.g., see K-Ras (G12D) Has a Potential Allosteric Small Molecule Binding Site, Feng H, Zhang Y, Bos P H, Chambers J M, Dupont M M, Stockwell B R, Biochemistry, 2019 May 28; 58 (21): 2542-2554. doi: 10.1021/acs.biochem.8b01300. Epub 2019 May 14; and Second harmonic generation detection of Ras conformational changes and discovery of a small molecule binder, Donohue E, Khorsand S, Mercado G, Varney K M, Wilder P T, Yu W, Mackerell A D Jr, Alexander P, Van Q N, Moree B, Stephen A G, Weber D J, Salafsky J, McCormick F., Proc Natl Acad Sci USA 2019 Aug. 27; 116 (35): 17290-17297, doi: 10.1073/pnas.1905516116. Epub 2019 Aug. 9), Clearly there remains a continued interest and effort to develop inhibitors of KRas, particularly inhibitors of activating KRas mutants, including KRas G12D.

While the KRas G12D inhibitors disclosed herein are potent inhibitors of KRas G12D signaling and exhibit single agent activity inhibiting the in vitro proliferation of cell lines harboring a KRas G12D mutation, the relative potency and/or observed maximal effect of any given KRas G12D inhibitor can vary between KRas mutant cell lines. The reason or reasons for the range of potencies and observed maximal effect is not fully understood but certain cell lines appear to possess differing intrinsic resistance. Thus, there is a need to develop alternative approaches to maximize the potency, efficacy, therapeutic index and/or clinical benefit of KRas G12D inhibitors in vitro and in vivo.

### Irinotecan

Irinotecan, sold under various beand names, is a chemotherapeutic cytotoxic agent approved for the treatment of colon cancer and small cell lung cancer. It is given intravenously, often with other chemotherapeutic agents. Irinotecan is activated by hydrolysis to SN-38, an inhibitor of topoisomerase I. This is then inactivated by glucuronidation by uridine diphosphate glucuronosyltransferase 1A1 (UGT1A1). The inhibition of topoisomerase I by the active metabolite SN-38 eventually leads to inhibition of both DNA replication and transcription. The molecular action of irinotecan occurs by trapping a subset of topoisomerase-1-DNA cleavage complexes, those with a guanine+1 in the DNA sequence. One irinotecan molecule stacks against the base pairs flanking the topoisomerase-induced cleavage site and poisons (inactivates) the topoisomerase 1 enzyme.

Irinotecan is a hydrophilic compound with a large volume of distribution (400 L/m2). At physiological pH, irinotecan and its active metabolite ethyl-10-hydroxy-camptothecin (SN-38) are present in two pH-dependent equilibrium isoforms; the anti tumor active lactone ring which hydrolyzed to the carboxylate isoform. In plasma, the majority of irinotecan and SN-38 are bound to albumin, which stabilizes their lactone forms. In blood, irinotecan and SN-38 are bound to platelets and red blood cells.

Irinotecan has a linear pharmacokinetic profile. Population pharmacokinetic models assumed a three-compartmental model for irinotecan and a two-compartmental model for SN-38. SN-38 has a short distribution half-life (approximately 8 min). It reached its peak plasma concentration within 2 h after infusion. Also SN-38 exhibit a second peak in the plasma concentration because of its enterohepatic re-circulation and its release from erythrocytes. About 2-5% of the pro-drug irinotecan is hydrolyzed into its active metabolite SN-38 in the liver by two carboxylesterase converting enzymes (CES1 and CES2) and in plasma by butyrylcholinesterase (hBChE). CES2 has a 12.5-fold higher affinity for irinotecan than CES1. While, butyrylcholinesterase has a 6-fold higher activity for irinotecan than CES. After conversion, SN-38 is actively transported to the liver by the organic anion transporting polypeptide (OATP) 1B1 transporter.

SN-38 is inactivated by glucuronidation to SN-38G (β-glucuronide conjugate) by several uridine diphosphate glucuronosyltransferase enzymes (UGTs) in the liver (UGT1A1, UGT1A9) and extra-hepatic (UGT1A1, UGT1A7, UGT1A10) and excreted into the bile. Several UGT polymorphisms affects irinotecan pharmacokinetics, for example, the decreased UGT1 activity, may lead to severe toxicity. Also, UGT1A1 conjugates bilirubin and bilirubin glucuronidation is another risk factor for increased toxicity.

### Other Chemotherapeutic Agents

Other chemotherapeutic agents used in the same manner or in a similar manner to irinotecan and irinotecan analogs incluse oxaliplatin, gemcitabine, docetaxel, 5 FU, pemetrexed, SN-38, abraxane and and nab-paclitaxel.

While irinotecan is a potent anti-cancer agent that exhibits activity alone and with other chemotherapeutic agents, the relative potency and/or observed maximal effect of irinotecan or irinotecan-based regimens can vary. The reason or reasons for such variation is not fully understood but certain cell lines appear to possess differing intrinsic resistance. Thus, there is a need to develop alternative approaches to maximize the potency, efficacy, therapeutic index and/or clinical benefit of irinotecan.

### SUMMARY OF THE INVENTION

The combination therapy of the present invention, in one aspect, increases the potency of KRas G12D inhibitors resulting in improved efficacy of KRas G12D inhibitors disclosed herein. The combination therapy of the present invention, in another aspect, provides improved clinical benefit to patients compared to treatment with KRas G12D inhibitors disclosed herein as a single agent.

Thus in one aspect of the invention there are provided therapeutically effective combinations of irinotecan:

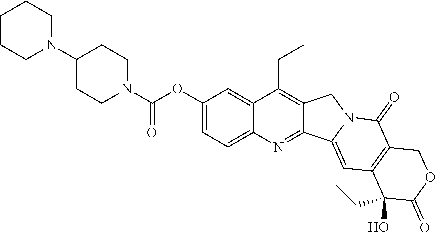 [SMILES: CCc1c2c(nc3ccc(OC(=O)N4CCC(N5CCCCC5)CC4)cc13)-c1cc3c(c(=O)n1C2)COC(=O)[C@]3(O)CC]

(S)-4,11-diethyl-3,4,12,14-tetrahydro-4-hydroxy-3,14-dioxo-1H-pyrano[3′,4′:6,7]indolizino[1,2-β]quinolin-9-yl ester[1,4′-bipiperidine]-1′-carboxylic acid trihydrate monohydrochloride

C₃₃H₃₈N₄O₆ Molecular Formula:

or a pharmaceutically acceptable salt thereof (most particularly the commercially available trihydrate monochloride form, typically depicted with ·3H₂0·HCl), and the KRas G12D inhibitor compound MRTX1133:

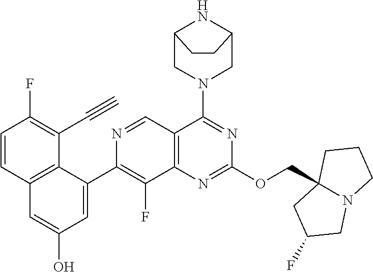 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

4-(4-((1R,5S)-3,8-diazabicyclo[3.2.1]octan-3-yl)-8-fluoro-2-(((2R,7aS)-2-fluorohexahydro-1Hpyrrolizin-7a-yl) methoxy)pyrido[4,3-d]pyrimidin-7-yl)-5-ethynyl-6-fluoronaphthalen-2-ol
   or a pharmaceutically acceptable salt thereof.

In another aspect of the invention there are provided therapeutically effective combinations of irinotecan or an irinotecan analog such as topotecan, belotecan, trastuzumab deruxtecan or camptothecin, or a pharmaceutically acceptable salt thereof, and the KRas G12D inhibitor MRTX1133, or a pharmaceutically acceptable salt thereof.

In another aspect of the invention, pharmaceutical compositions are provided for use in the methods comprising a therapeutically effective amount of a combination of irinotecan or an irinotecan analog or a pharmaceutically acceptable salt thereof, and the KRas G12D inhibitor compound MRTX1133 or MRTX1133 analogs and related compounds such as any of the compounds disclosed and described in WIPO publication WO2021/041671, including but not limited to: Ex. 252 (MRTX1133), 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol; Ex. 243, 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; Ex. 246, 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; Ex. 251, 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; Ex. 253, 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; Ex. 259, 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and Ex. 282, 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; or a pharmaceutically acceptable salt thereof and a pharmaceutically acceptable excipient.

In one aspect of the invention, provided herein are methods of treating cancer in a subject in need thereof, comprising administering to the subject a therapeutically effective amount of a combination of irinotecan or a pharmaceutically acceptable salt thereof and the KRas G12D inhibitor MRTX1133 or a pharmaceutically acceptable salt or a pharmaceutical composition thereof.

In another aspect of the invention, provided herein are methods of treating cancer in a subject in need thereof, comprising administering to the subject a therapeutically effective amount of a combination of irinotecan or an irinotecan analog or a pharmaceutically acceptable salt thereof, and the KRas G12D inhibitor MRTX1133 or a MRTX1133 analog or a pharmaceutically acceptable salt or a pharmaceutical composition thereof.

In one embodiment, the cancer is a KRas G12D-associated cancer. In one embodiment, the KRas G12D-associated cancer is pancreatic, colorectal, endometrial, and non-small cell lung cancer.

In some aspects of the invention, irinotecan and the KRas G12D inhibitor compound (such as MRTX1133) are the only active agents in the provided compositions and methods.

Besides irinotecan or a pharmaceutically acceptable salt thereof, examples of irinotecan analogs suitable for the provided compositions and methods include, but are not limited to topotecan, belotecan, trastuzumab deruxtecan and camptothecin or a pharmaceutically acceptable salt thereof.

Chemotherapeutic agents besides irinotecan and irinotecan analogs that can be effectively used in combination with MRTX1133 or MRTX1133 analogs or their salts include: oxaliplatin, gemcitabine, docetaxel, 5 FU, pemetrexed, SN-38, abraxane, paclitaxel and nab-paclitaxel. Also included is gemcitabine used in combination with nab-paclitaxel (and in further combination with MRTX1133 or MRTX1133 analogs or their salts).

Besides MRTX1133, examples of KRas G12D inhibitors suitable for the provided compositions and methods include, but are not limited to: 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorobexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-S-fluoronaphthalen-2-ol; and pharmaceutically acceptable salts thereof.

In yet another aspect, the invention provides for methods for increasing the sensitivity of a cancer cell to a KRas G12D inhibitor, comprising contacting the cancer cell with a therapeutically effective amount of a combination of irinotecan (or irinotecan analog) or a pharmaceutically acceptable salt thereof and a KRas G12D inhibitor compound such as MRTX1133 (or a MRTX1133 analog) or a pharmaceutically acceptable salt or a pharmaceutical composition thereof, wherein the irinotecan or irinotecan analog (or salt) increases the sensitivity of the cancer cell to the KRas G12D inhibitor. In one embodiment, the contacting is in vitro. In one embodiment, the contacting is in vivo.

Also provided herein are methods for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of irinotecan or an irinotecan analog or a pharmaceutically acceptable salt thereof, and a KRas G12D inhibitor such as MRTX1133 or a MRTX1133 analog, or a pharmaceutically acceptable salt or a pharmaceutical composition thereof, wherein irinotecan or the irinotecan analog or a pharmaceutically acceptable salt thereof increases the sensitivity of the KRas G12D-associated cancer to MRTX1133 or a MRTX1133 analog.

Also provided herein are kits comprising irinotecan or an irinotecan analog or a pharmaceutically acceptable salt thereof and the KRas G12D inhibitor compound MRTX1133 or a MRTX1133 analog, or a pharmaceutically acceptable salt or a pharmaceutical composition thereof. Also provided is a kit comprising irinotecan or an irinotecan analog or a pharmaceutically acceptable salt thereof, and the KRas G12D inhibitor compound MRTX1133 or a MRTX1133 analog, or a pharmaceutically acceptable salt or a pharmaceutical composition thereof, for use in treating a KRas G12D cancer.

In a related aspect, the invention provides a kit containing a dose of irinotecan or an irinotecan analog or a pharmaceutically acceptable salt thereof, and the KRas G12D inhibitor compound MRTX1133 or a MRTX1133 analog or a pharmaceutically acceptable salt or a pharmaceutical composition thereof, in an amount effective to inhibit proliferation of cancer cells in a subject. The kit in some cases includes an insert with instructions for administration of irinotecan or an irinotecan analog or a pharmaceutically acceptable salt thereof, and the KRas 012D inhibitor compound MRTX1133 or a MRTX1133 analog or a pharmaceutically acceptable salt or a pharmaceutical composition thereof. The insert may provide a user with one set of instructions for using the irinotecan or an irinotecan analog or a pharmaceutically acceptable salt thereof, in combination with the KRas G12D inhibitor compound MRTX1133 or a MRTX1133 analog or a pharmaceutically acceptable salt or a pharmaceutical composition thereof.

In some aspects of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 depicts the average tumor volumes in mouse xenografts for MRTX1133, alone and in combination with irinorecan (LS180 colon cancer cell line).

FIG. 2 depicts the average tumor volumes in mouse xenografts for MRTX1133, alone and in combination with irinorecan (PANC0203 pancreatic cancer cell line).

FIG. 3 depicts the average tumor volumes in mouse xenografts for MRTX1133, alone and in combination with irinorecan (SNU1033 rectal cancer cell line).

### DETAILED DESCRIPTION OF THE INVENTION

The present invention relates to combination therapies for treating KRas G12D cancers. In particular, the present invention relates to methods of treating cancer in a subject in need thereof, comprising administering to the subject a therapeutically effective amount of irinotecan or irinotecan analog or a pharmaceutically acceptable salt thereof (“irinotecans”), and the KRas G12D inhibitor MRTX1133 or MRTX1133 analog, or a pharmaceutically acceptable salt or a pharmaceutical composition thereof, pharmaceutical compositions comprising therapeutically effective amounts of the two agents, kits comprising the compositions and methods of use thereof.

Combinations of irinotecan, analogs thereof or a pharmaceutically acceptable salt thereof with a KRas G12D inhibitor such as MRTX1133 or MRTX1133 analog, or a pharmaceutically acceptable salt or a pharmaceutical composition thereof, increase the potency of the KRas G12D inhibitor compound against cancer cells that express KRas G12D thereby increasing the efficacy and therapeutic index of the KRas G12D inhibitor compound or pharmaceutically acceptable salts thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Gly12Asp.

As used herein, a “KRas G12D inhibitor” refers to compounds such as those represented and depected in WO2021/041671, or pharmaceutically acceptable salts thereof, as well as in other publications. These compounds are capable of negatively modulating or inhibiting all or a portion of the enzymatic activity of KRas G12D. The KRas G12D inhibitors of the present invention interact with and non-covalently bind to KRas G12D in the switch II pocket and inhibit protein-protein interactions necessary for activation of the KRAS pathway. MRTX1133 is an example of a KRas G12D inhibitor.

A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, “irinotecan” refers to the compound:

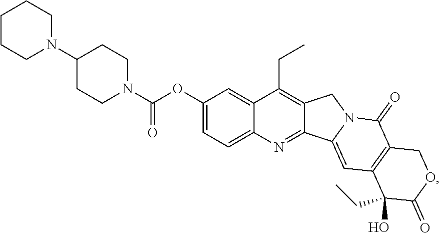 [SMILES: CCc1c2c(nc3ccc(OC(=O)N4CCC(N5CCCCC5)CC4)cc13)-c1cc3c(c(=O)n1C2)COC(=O)[C@]3(O)CC]

most typically the trihydrate monochloride form, but also to other salt and/or hydrated and/or solvated forms.

As used herein, a “irinotecan analog” refers to a compound that is structurally related to irinotecan, such as topotecan, belotecan, trastuzumab deruxtecan or camptothecin.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12D mutation (e.g., as determined using a regulatory agency-approved, e.g., FDA-approved, assay or kit). In some embodiments, the subject has a tumor that is positive for a KRas G12D mutation (e.g., as determined using a regulatory agency-approved assay or kit). The subject can be a subject with a tumor(s) that is positive for a KRas G12D mutation (e.g., identified as positive using a regulatory agency-approved, e.g., FDA-approved, assay or kit). The subject can be a subject whose tumors have a KRas G12D mutation (e.g., where the tumor is identified as such using a regulatory agency-approved, e.g., FDA-approved, kit or assay). In some embodiments, the subject is suspected of having a KRas G12D gene-associated cancer. In some embodiments, the subject has a clinical record indicating that the subject has a tumor that has a KRas G12D mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

In some embodiments of any of the methods or uses described herein, an assay is used to determine whether the patient has KRas G12D mutation using a sample (e.g., a biological sample or a biopsy sample such as a paraffin-embedded biopsy sample) from a patient (e.g., a patient suspected of having a KRas G12D-associated cancer, a patient having one or more symptoms of a KRas G12D-associated cancer, and/or a patient that has an increased risk of developing a KRas G12D-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

As used herein, “an effective amount” of a compound is an amount that is sufficient to negatively modulate or inhibit the activity of the desired target, or otherwise arrest or slow proliferation of the targeted cells, i.e., irinotecan or KRas G12D. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of KRas G12D. Such amount may be administered as a single dosage or may be administered according to a regimen, whereby it is effective.

As used herein, a “therapeutically effective amount of a combination” of two compounds is an amount that together increases the activity of the combination in comparison to the therapeutically effective amount of each compound in the combination, i.e., more than merely additive. Alternatively, in vivo, the therapeutically effective amount of the combination of irinotecan or an irinotecan analog or a pharmaceutically acceptable salt thereof, and the KRas G12D inhibitor compound MRTX1133 or MRTX1133 analog, or a pharmaceutically acceptable salt or a pharmaceutical composition thereof, results in an increased duration of overall survival (“OS”) in subjects relative to treatment with only the KRas G12D inhibitor. In one embodiment, the therapeutically effective amount of the combination of irinotecan or an irinotecan analog or a pharmaceutically acceptable salt thereof, and the KRas G12D inhibitor compound MRTX1133 or MRTX1133 analog, or a pharmaceutically acceptable salt or a pharmaceutical composition thereof, results in an increased duration of progression-free survival (“PFS”) in subjects relative to treatment with only the KRas G12D inhibitor. In one embodiment, the therapeutically effective amount of the combination of irinotecan or an irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in increased tumor regression in subjects relative to treatment with only the KRas G12D inhibitor. In one embodiment, the therapeutically effective amount of the combination of irinotecan or an irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in increased tumor growth inhibition in subjects relative to treatment with only the KRas G12D inhibitor. In one embodiment, the therapeutically effective amount of the combination of irinotecan or an irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in an improvement in the duration of stable disease in subjects compared to treatment with only the KRas G12D inhibitor. The amount of each compound in the combination may be the same or different than the therapeutically effective amount of each compound when administered alone as a monotherapy. Such amounts may be administered as a single dosage or may be administered according to a regimen, whereby it is effective.

As used herein, “treatment” means any manner in which the symptoms or pathology of a condition, disorder or disease are ameliorated or otherwise beneficially altered. Treatment also encompasses any pharmaceutical use of the compositions herein.

As used herein, “amelioration” of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a KRas inhibitor or a pharmaceutically acceptable salt thereof, or the dose of irinotecan, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### KRas G12D Inhibitor Compounds

In one aspect of the invention, provided herein are methods of treating cancer in a subject in need thereof, comprising administering to the subject a therapeutically effective amount of a combination of irinotecan or an irinotecan analog or a pharmaceutically acceptable salt thereof, and the KRas G12D inhibitor compound MRTX1133 or a pharmaceutically acceptable salt or a pharmaceutical composition thereof.

In one embodiment, the KRas G12D inhibitor is:

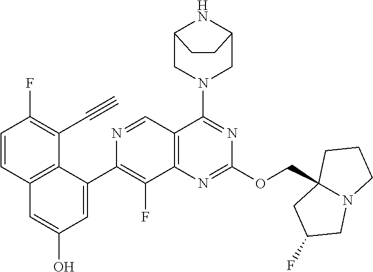 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

(also referred to as MRTX1133, and Eaxmple 252 in WO2021/041671) or a pharmaceutically acceptable salt thereof.

The KRas G12D inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12D inhibitor compound MRTX1133 used in the methods include salts of the above compounds, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing the KRas G12D inhibitors disclosed herein are known. For example, WO2021/041671 describes general reaction schemes for preparing compounds including MRTX1133 and MRTX1133 analogs, and also provide detailed synthetic routes for the preparation of these compounds.

### Irinotecan and Irinotecan Analogs

In one embodiment, the irinotecan or irinotecan analog of the present invention is Irinotecan:

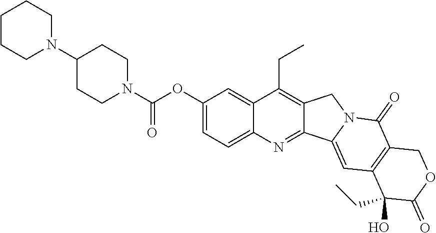 [SMILES: CCc1c2c(nc3ccc(OC(=O)N4CCC(N5CCCCC5)CC4)cc13)-c1cc3c(c(=O)n1C2)COC(=O)[C@]3(O)CC]

In one embodiment, irinotecan is the trihydrate monochloride form, typically depicted with ·3H₂O·HCl.

In one embodiment, the irinotecan or irinotecan analog of the present invention is the irinotecan analog topotecan:

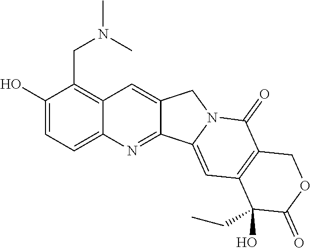 [SMILES: CC[C@@]1(O)C(=O)OCc2c1cc1n(c2=O)Cc2cc3c(CN(C)C)c(O)ccc3nc2-1]

or a pharmaceutically acceptable salt thereof.

In one embodiment, the irinotecan or irinotecan analog of the present invention is the irinotecan analog belotecan:

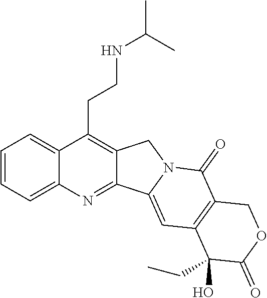 [SMILES: CC[C@@]1(O)C(=O)OCc2c1cc1n(c2=O)Cc2c-1nc1ccccc1c2CCNC(C)C]

or a pharmaceutically acceptable salt thereof.

In one embodiment, the irinotecan or irinotecan analog of the present invention is the irinotecan analog trastuzumab-deruxtecan:

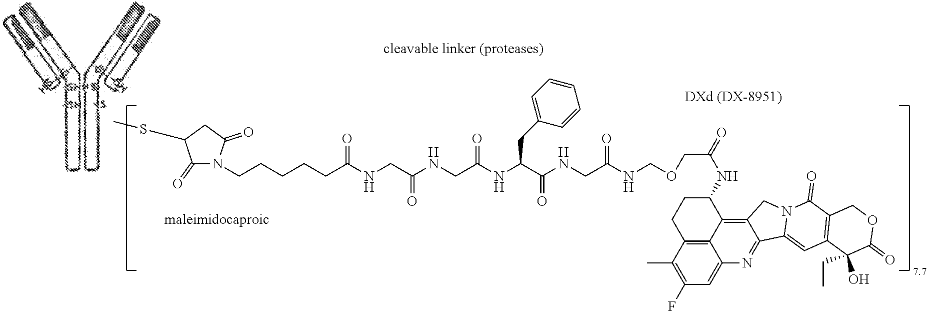

or a pharmaceutically acceptable salt thereof.

In one embodiment, the irinotecan or irinotecan analog of the present invention is the irinotecan analog camptothecin:

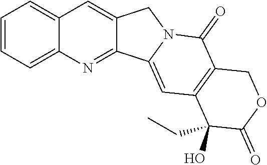 [SMILES: CC[C@@]1(O)C(=O)OCc2c1cc1n(c2=O)Cc2cc3ccccc3nc2-1]

or a pharmaceutically acceptable salt thereof.

Irinotecan and irinotecan analogs used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CIIIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diasteromeric) and racemic forms.

In another embodiment, the recited irinotecan and irinotecan analogs include their saltss, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Methods for manufacturing irinotecan and irinotecan analogs are well known, and these compounds are commercially available.

### Pharmaceutical Compositions

Irinotecan or irinotecan analogs or a pharmaceutically acceptable salt thereof, and the KRas G12D compound MRTX1133 or MRTX1133 analogs, or pharmaceutically acceptable salts thereof, may be formulated into pharmaceutical compositions.

In another aspect, the invention provides pharmaceutical compositions comprising irinotecan or an irinotecan analog or a pharmaceutically acceptable salt thereof, and the KRas G12D compound MRTX1133 or MRTX1133 analogs, or pharmaceutically acceptable salts thereof, and one or more of a pharmaceutically acceptable carrier, excipient, or diluent that may be used in the methods disclosed herein. Irinotecan or irinotecan analogs or a pharmaceutically acceptable salt thereof, and the KRas G12D compound MRTX1133 or MRTX1133 analogs, or pharmaceutically acceptable salts thereof, may be independently formulated by any method well known in the art and may be prepared for administration by any route, including, without limitation, parenteral, oral, sublingual, transdermal, topical, intranasal, intratracheal, intravenous or intrarectal. In certain embodiments, the two aforementioned components are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising irinotecan or irinotecan analogs or a pharmaceutically acceptable salt thereof, and the KRas G12D compound MRTX1133 or MRTX1133 analogs, or pharmaceutically acceptable salts thereof, or a pharmaceutical composition thereof, may be used in the methods of use described herein.

### Co-Administration

The irinotecan or irinotecan analogs or a pharmaceutically acceptable salt thereof, and the KRas G12D compound MRTX1133 or MRTX1133 analogs, or a pharmaceutically acceptable salt thereof, can be formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising irinotecan or irinotecan analogs or a pharmaceutically acceptable salt thereof, and the KRas G12D compound MRTX1133 or MRTX1133 analogs, or a pharmaceutically acceptable salt thereof, for use in the methods may be for simultaneous, separate or sequential use. In one embodiment, irinotecan or irinotecan analog or a pharmaceutically acceptable salt thereof is administered prior to administration of the KRas G12D compound MRTX1133 or MRTX1133 analog, or pharmaceutically acceptable salt thereof. In another embodiment, irinotecan or irinotecan analog or a pharmaceutically acceptable salt thereof is administered after administration of the KRas G12D compound MRTX1133 or MRTX1133 analog or a pharmaceutically acceptable salt thereof. In another embodiment, irinotecan or irinotecan analog or a pharmaceutically acceptable salt thereof is administered at about the same time as administration of the KRas G12D compound MRTX1133 or MRTX1133 analog or pharmaceutically acceptable salt thereof.

Separate administration of each inhibitor, at different times and by different routes, in some cases would be advantageous. Thus, the components in the combination, i.e., irinotecan or irinotecan analog or a pharmaceutically acceptable salt thereof, and the KRas G12D compound MRTX1133 or MRTX1133 analogs, or pharmaceutically acceptable salt thereof, need not be necessarily administered at essentially the same time or in any order.

Oncology drugs are typically administered at the maximum tolerated dose (“MTD”), which is the highest dose of drug that does not cause unacceptable side effects. In one embodiment, irinotecan or irinotecan analog or a pharmaceutically acceptable salt thereof, and the KRas G12D compound MRTX1133 or MRTX1133 analog, or a pharmaceutically acceptable salt or a pharmaceutical composition thereof, are each dosed at their respective MTDs. In one embodiment, irinotecan or irinotecan analog or a pharmaceutically acceptable salt or a pharmaceutical composition thereof is dosed at its MTD, and the KRas G12D compound MRTX1133 or MRTX1133 analog, or a pharmaceutically acceptable salt or a pharmaceutical composition thereof, is dosed in an amount less than its MTD. In one embodiment, irinotecan or irinotecan analog or a pharmaceutically acceptable salt thereof is dosed at an amount less than its MTD and the KRas G12D compound MRTX1133 or MRTX1133 analog, or a pharmaceutically acceptable salt thereof is dosed at its MTD. In one embodiment, the both components are each dosed at less than their respective MTDs. The administration can be so timed that the peak pharmacokinetic effect of one compound coincides with the peak pharmacokinetic effect of the other.

In one embodiment, a single dose of KRas G12D inhibitor compound MRTX1133 or MRTX1133 analog, or a pharmaceutically acceptable salt or a pharmaceutical composition thereof, is administered per day (i.e., in about 24 hour intervals) (i.e., QD). In another embodiment, two doses of KRas G12D inhibitor compound MRTX1133 or MRTX1133 analog, or a pharmaceutically acceptable salt or a pharmaceutical composition thereof, are administered per day (i.e., BID). In another embodiment, three doses of KRas G12D inhibitor compound MRTX1133 or MRTX1133 analog or a pharmaceutically acceptable salt or a pharmaceutical composition thereof, are administered per day (i.e., TID).

In one embodiment, the irinotecan or irinotecan analog or a pharmaceutically acceptable salt thereof, is administered QD. In another embodiment irinotecan or irinotecan analog or a pharmaceutically acceptable salt thereof, is administered BID. In another embodiment irinotecan or irinotecan analog or a pharmaceutically acceptable salt thereof of the invention are administered TID.

In one embodiment, a single dose of irinotecan or irinotecan analog or a pharmaceutically acceptable salt thereof, and KRas G12D inhibitor compound MRTX1133 or MRTX1133 analog or a pharmaceutically acceptable salt or a pharmaceutical composition thereof are each administered once daily.

Examples of irinotecan and irinotecan analogs or a pharmaceutically acceptable salt thereof suitable for the provided compositions and methods include those mentioned herein, for example: irinotecan, topotecan, belotecan, trastuzumab deruxtecan and camptothecin.

Examples of KRas G12D inhibitors suitable for the provided compositions and methods include those mentioned herein, for example: MRTX1133: 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; or a pharmaceuticallyacceptable salt thereof and a pharmaceutically acceptable excipient.

### Combination Therapies

In one aspect of the invention, provided herein are methods of treating cancer in a subject in need thereof, comprising administering to the subject a therapeutically effective amount of a combination of irinotecan or an irinotecan analog or a pharmaceutically acceptable salt thereof, and the KRas G12D inhibitor compound MRTX1133 or an MRTX1133 analog, or a pharmaceutically acceptable salt or a pharmaceutical composition thereof. In one embodiment, the cancer is a KRas G12D-associated cancer. In one embodiment, the KRas G12D-associated cancer is pancreatic, colorectal, endometrial, and non-small cell lung cancers.

In yet another aspect, the invention provides for methods for increasing the sensitivity of a cancer cell to a KRas G12D inhibitor, comprising contacting the cancer cell with an effective amount of a combination of irinotecan or irinotecan analog or a pharmaceutically acceptable salt thereof, and the KRas G12D inhibitor compound MRTX1133 or an MRTX1133 analog, or a pharmaceutically acceptable salt or a pharmaceutical composition thereof, wherein irinotecan or irinotecan analog or a pharmaceutically acceptable salt thereof increases the sensitivity of the cancer cell to the KRas G12D inhibitor. In one embodiment, the contacting is in vitro. In one embodiment, the contacting is in vivo.

In one embodiment, the combination therapy comprises a combination of a compound having the formula:

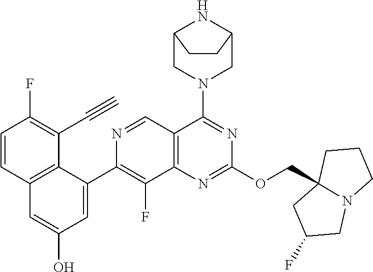 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

and irinotecan or an irinotecan analog or a pharmaceutically acceptable salt thereof.

In one embodiment, the combination therapy comprises a combination of a compound having the formula:

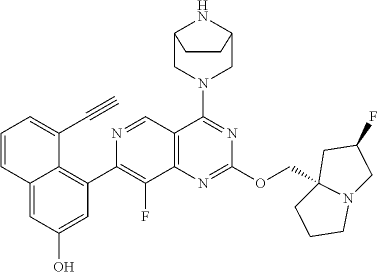 [SMILES: C#Cc1cccc2cc(O)cc(-c3ncc4c(N5CC6CCC(C5)N6)nc(OC[C@@]56CCCN5C[C@H](F)C6)nc4c3F)c12]

and irinotecan or an irinotecan analog or a pharmaceutically acceptable salt thereof.

In one embodiment, the combination therapy comprises a combination of a compound having the formula:

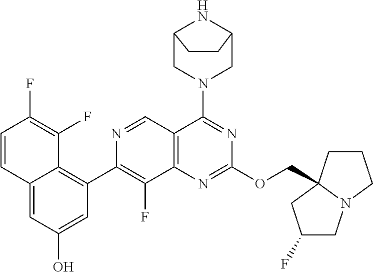 [SMILES: Oc1cc(-c2ncc3c(N4CC5CCC(C4)N5)nc(OC[C@@]45CCCN4C[C@H](F)C5)nc3c2F)c2c(F)c(F)ccc2c1]

and irinotecan or an irinotecan analog or a pharmaceutically acceptable salt thereof.

In one embodiment, the combination therapy comprises a combination of a compound having the formula:

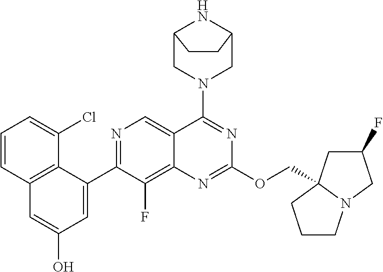 [SMILES: Oc1cc(-c2ncc3c(N4CC5CCC(C4)N5)nc(OC[C@@]45CCCN4C[C@H](F)C5)nc3c2F)c2c(Cl)cccc2c1]

and irinotecan or an irinotecan analog or a pharmaceutically acceptable salt thereof.

In one embodiment, the combination therapy comprises a combination of a compound having the formula:

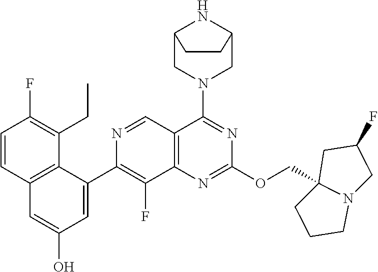 [SMILES: CCc1c(F)ccc2cc(O)cc(-c3ncc4c(N5CC6CCC(C5)N6)nc(OC[C@@]56CCCN5C[C@H](F)C6)nc4c3F)c12]

and irinotecan or an irinotecan analog or a pharmaceutically acceptable salt thereof.

In one embodiment, the combination therapy comprises a combination of a compound having the formula:

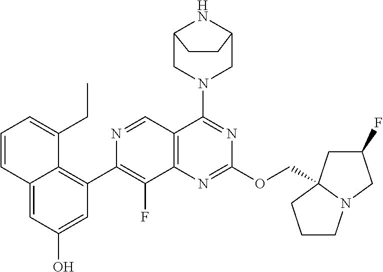 [SMILES: CCc1cccc2cc(O)cc(-c3ncc4c(N5CC6CCC(C5)N6)nc(OC[C@@]56CCCN5C[C@H](F)C6)nc4c3F)c12]

and irinotecan or an irinotecan analog or a pharmaceutically acceptable salt thereof.

In one embodiment, the combination therapy comprises a combination of a compound having the formula:

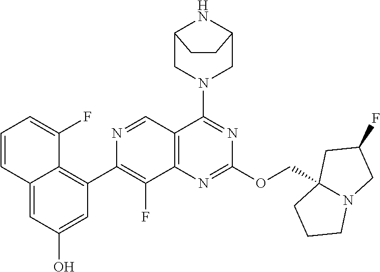 [SMILES: Oc1cc(-c2ncc3c(N4CC5CCC(C4)N5)nc(OC[C@@]45CCCN4C[C@H](F)C5)nc3c2F)c2c(F)cccc2c1]

and irinotecan or an irinotecan analog or a pharmaceutically acceptable salt thereof.

In one embodiment, the irinotecan or an irinotecan analog or a pharmaceutically acceptable salt thereof referred to above is irinotecan.

In one embodiment, the irinotecan or an irinotecan analog or a pharmaceutically acceptable salt thereof referred to above is topotecan.

In one embodiment, the irinotecan or an irinotecan analog or a pharmaceutically acceptable salt thereof referred to above is belotecan.

In one embodiment, the irinotecan or an irinotecan analog or a pharmaceutically acceptable salt thereof referred to above is trastuzumab deruxtecan.

In one embodiment, the irinotecan or an irinotecan analog or a pharmaceutically acceptable salt thereof referred to above is camptothecin.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12D mutation, as well as, for example, introducing a combination provided herein into a sample containing a cellular or purified preparation containing KRas G12D mutation.

By negatively modulating the activity of KRas G12D, the methods described herein are designed to inhibit undesired cellular proliferation resulting from enhanced KRas G12D activity within the cell. The ability of a compound to inhibit KRas G12D may be monitored in vitro using well known methods, including those described in published international PCT application WO2021/041671. Likewise, the inhibitory activity of combination in cells may be monitored, for example, by measuring the inhibition of KRas G12D activity of the amount of phosphorylated ERK to assess the effectiveness of treatment and dosages may be adjusted accordingly by the attending medical practitioner.

The compositions and methods provided herein may be used for the treatment of a KRas G12D-associated cancer in a subject in need thereof, comprising administering to said subject a therapeutically effective amount of a combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, wherein the irinotecan or irinotecan analog or a pharmaceutically acceptable salt thereof increases the sensitivity the KRas G12D-associated cancer to the KRas G12D inhibitor. In one embodiment, the KRas G12D-associated cancer is colorectal cancer. In one embodiment, the KRas G12D-associated cancer is pancreatic cancer. In one embodiment, the KRas G12D-associated cancer is endometrial cancer. In one embodiment, the KRas G12D-associated cancer is non small cell lung cancer.

In one embodiment, the therapeutically effective amount of the combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in an increased duration of overall survival (“OS”) in subjects relative to treatment with only the KRas G12D inhibitor. In one embodiment, the therapeutically effective amount of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in an increased duration of progression-free survival (“PFS”) in subjects relative to treatment with only the KRas G12D inhibitor. In one embodiment, the therapeutically effective amount of the combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in increased tumor regression in subjects relative to treatment with only the KRas G12D inhibitor. In one embodiment, the therapeutically effective amount of the combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in increased tumor growth inhibition in subjects relative to treatment with only the KRas G12D inhibitor. In one embodiment, the therapeutically effective amount of the combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, results in an improvement in the duration of stable disease in subjects compared to treatment with only the KRas G12D inhibitor.

In another embodiment, irinotecan or irinotecan analog or a pharmaceutically acceptable salt thereof is administeredin combination wth the KRas G12D inhibitor compound MRTX1133 or MRTX1133 analog or a pharmaceutically acceptable salt or a pharmaceutical composition thereof, once disease progression has been observed for KRas G12D monotherapy, in which the combination therapy results in enhanced clinical benefit for the patient by increasing OS, PFS, tumor regression, tumor growth inhibition or the duration of stable disease in the patient. In one embodiment, the KRas G12D inhibitor is a compound selected from MRTX1133 and MRTX1133 analogs such as 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; and pharmaceutically acceptable salts thereof.

In one embodiment, the therapeutic combination comprises therapeutically effective amounts of MRTX1133 or a pharmaceutically acceptable salt thereof and irinotecan or a pharmaceutically acceptable salt thereof.

In one embodiment, the therapeutic combination comprises therapeutically effective amounts of MRTX1133 or a pharmaceutically acceptable salt thereof and topotecan or a pharmaceutically acceptable salt thereof.

In one embodiment, the therapeutic combination comprises therapeutically effective amounts of MRTX1133 or a pharmaceutically acceptable salt thereof and belotecan or a pharmaceutically acceptable salt thereof.

In one embodiment, the therapeutic combination comprises therapeutically effective amounts of MRTX1133 or a pharmaceutically acceptable salt thereof and, trastuzumab deruxtecan or a pharmaceutically acceptable salt thereof.

In one embodiment, the therapeutic combination comprises therapeutically effective amounts of MRTX1133 or a pharmaceutically acceptable salt thereof and and camptothecin or a pharmaceutically acceptable salt thereof.

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with a KRas G12D mutation (e.g., a KRas G12D-associated cancer) (e.g., as determined using a regulatory agency-approved, e.g., FDA-approved, assay or kit); and (b) administering to the patient a therapeutically effective amount of a combination of irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, wherein irinotecan or irinotecan analog or a pharmaceutically acceptable salt thereof increases the sensitivity of the KRas G12D-associated cancer to the KRas G12D inhibitor. In one embodiment, the KRas G12D inhibitor is a compound selected from MRX-'1133 and MRTX1133 analogs such as 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7a8)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; and pharmaceutically acceptable salts thereof.

In one embodiment, irinotecan or irinotecan analog or a pharmaceutically acceptable salt thereof such as topotecan, belotecan, trastuzumab deruxtecan or camptothecin is employed.

In one embodiment, the therapeutic combination comprises therapeutically effective amounts of irinotecan or a pharmaceutically acceptable salt thereof.

In a further embodiment, the therapeutic combination comprises therapeutically effective amounts of topotecan or a pharmaceutically acceptable salt thereof.

In a further embodiment, the therapeutic combination comprises therapeutically effective amounts of belotecan or a pharmaceutically acceptable salt thereof.

In a further embodiment, the therapeutic combination comprises therapeutically effective amounts of trastuzumab deruxtecan or a pharmaceutically acceptable salt thereof.

In a further embodiment, the therapeutic combination comprises therapeutically effective amounts of or camptothecin or a pharmaceutically acceptable salt thereof.

In a further embodiment, the therapeutic combination comprises therapeutically effective amounts of MRTX1133.

In a further embodiment, the therapeutic combination comprises therapeutically effective amounts of 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; or pharmaceutically acceptable salts thereof.

In one embodiment, the KRas G12D MRTX1133 or a pharmaceutically acceptable salt thereof, is administered as a parenteral, oral, sublingual, transdermal, topical, intranasal, intratracheal, intravenous or intrarectal formulation during a period of time. In one embodiment, the dose of MRTX1133 adminitered comprises on or more of: about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg and about 2000 mg. In one embodiment, MRTX1133 is administered once a day (QD) on a daily basis during a period of time. In one embodiment MRTX1133 is administered twice a day (BID) on a daily basis during a period of time.

In one embodiment, itinotecan or irinotecan analog or a pharmaceutically acceptable salt thereof is intravenously administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg), over a period of time.

In one embodiment, irinotecan is intravenously administered via 30- or 90-minite infusions of 125 mg/m² weekly for four of every six weeks, or 350 mg/m² every three weeks.

In another embodiment, irinotecan is intravenously administered via 30- or 90-minite infusions of less than 125 mg/m² weekly for four of every six weeks, or less than 350 mg/m² every three weeks.

In one embodiment, irinotecan is intravenously infused as directed on the CAMPTOSAR® product label.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to, and/or provides, a kit comprising irinotecan or irinotecan analog or a pharmaceutically acceptable salt thereof, and the KRas G12D inhibitor compound MRTX1133 or MRTX1133 analog or a pharmaceutically acceptable salt or a pharmaceutical composition thereof, for use in treating a cancer.

In a related aspect, the invention provides a kit containing a dose of irinotecan or irinotecan analog or a pharmaceutically acceptable salt thereof, and dose of the KRas G12D inhibitor compound MRTX1133 or MRTX1133 analog or a pharmaceutically acceptable salt or a pharmaceutical composition thereof, in an amount effective to inhibit proliferation of cancer cells, particularly KRas G12D-expressing cancer cells, in a subject. The kit in some cases includes an insert with instructions for administration of theses agents, where the insert may provide a user with one set of instructions for using these agents in combination.

The following Examples are intended to illustrate further certain embodiments of the invention and are not intended to limit the scope of the invention.

### Example A

In Vivo Models for Examination of KRas G12D inhibitor Plus Irinorecan Combinations

Immunocompromised nude/nude mice are inoculated in the right hind flank with cells harboring a KRas G12D mutation. When tumor volumes reach between 200-400 mm³ in size, the mice are divided into four to five groups of 4-5 mice each. The first group is administered vehicle only. The second group is administered a twice daily single agent dose of the KRas G12D inhibitor at a concentration that yields a maximal biological effect or a less than maximal biological effect, depending on the cell line and the single agent activity, that does not result in complete tumor regression. The second group, depending on cell line, may be administered a twice daily for 2 sequential days followed by 5 days off, the KRas G12D inhibitor at a concentration that yields a maximal biological effect or a less than maximal biological effect, depending on the cell line and single agent activity, that does not result in complete tumor regression. The third group is administered a single agent dose of irinotecan at a concentration that yields a maximal biological effect or a less than maximal biological effect, depending on the cell line and the single agent activity, that also does not result in complete tumor regression. The fourth group is administered the single agent dose of the KRas G12D inhibitor using the twice daily for 2 sequential days followed by 5 days off schedule in combination with the single agent dose of Irinotecan. The treatment period varies from cell line to cell line but typically is between 15-22 days. Tumor volumes are measured using a caliper every two-three days and tumor volumes are calculated by the formula: 0.5×(Length×Width)². A greater degree of tumor growth inhibition for the combination in this model demonstrates that the combination therapy is likely to have a clinically meaningful benefit to treated subjects relative to treatment with only a KRas G12D inhibitor.

25 nude/nude micewere inoculated in the right hind limb with 5×10⁶ LS180 cells. 20 nude/nude mice were inoculated in the right hind limb with 5×10⁶ Panc 02.03 cells. 20 nude/nude mice were inoculated in the right hind limb with 5×10⁶ SNU-1033 cells. For all models, when tumor volume reached ˜200-400 mm³ (Study Day 0), 5 mice in each of the groups were administered i.p.: vehicle only (10% captisol in 50 mM citrate buffer, pH 5.0), 30 mg/kg of KRas G12D inhibitor MRTX1133 (10% Captisol in 50 mM citrate buffer, pH 5.0), 100 mg/kg Irinotecan (saline vehicle), or 30 mg/kg of KRas G12D inhibitor MRTX1133 and 100 mg/kg of Irinotecan. MRTX1133 was treated either i.p. twice daily for the duration of the study or i.p. twice daily for 2 consecquetive days followed by days off. Irinotecan was dosed i.p. once every 7 days for the duration of the study. Tumor volumes, measured at pre-specified days, for the five mice per group were averaged and are reported for each xenograft model in the tables provided below.

### Example B

KRas G12D Inhibitor MRTX1133 in Combination with Irinorecan (LS180 Colon Cancer Cell Line)

25 nude/nude mice were inoculated with LS180 cells in the hind right flank. When the tumors reached ˜250 mm³ five treatment groups were established with 5 mice per group. The results of this study are provided in Table 1:

TABLE 1
Average Tumor Volumes (mm³) of nude/nude Tumor Bearing
Mice Treated with Single Agents and in Combination

| | Vehicle | MRTX1133 | MRTX1133 | Irinotecan | Irino + |
|---|---|---|---|---|---|
| Study | BID | 30 mg/kg | 30 mg/kg | 100 mg/kg | MRTX1133 |
| Day | Daily | BID Daily | BID 2x/week | Q7D | 2x/week |
| 1 | 268.842 | 268.94 | 272.338 | 257.606 | 278.622 |
| 4 | 601.218 | 418.808 | 570.528 | 430.938 | 347.424 |
| 8 | 1089.326 | 593.71 | 1134.668 | 490.91 | 447.342 |
| 11 | 1738.074 | 808.808 | 1439.99 | 594.972 | 493.694 |
| 15 | 1965.054 | 1197.106 | 1899.832 | 653.082 | 554.318 |

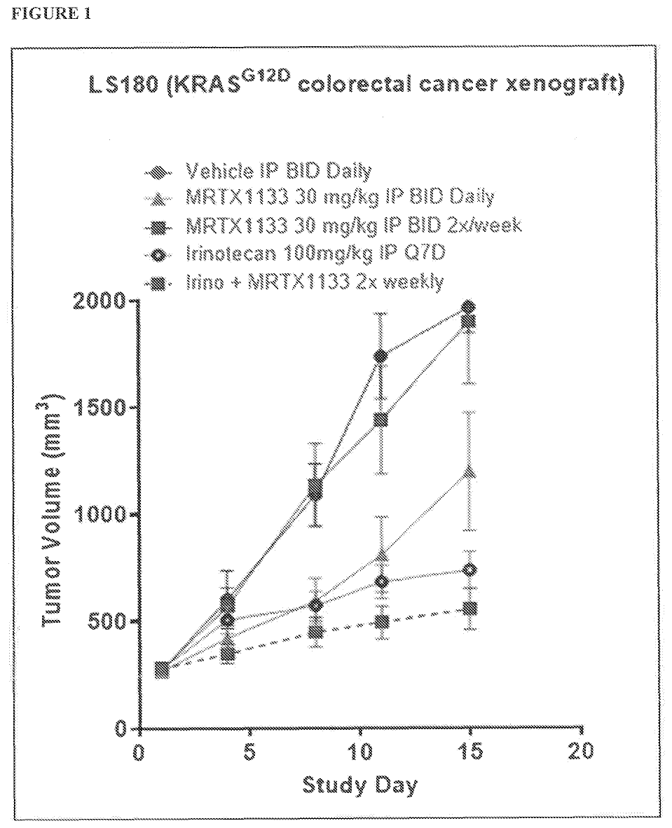

As shown in Table 1, the administration of MRTX1133 as a single agent dosed twice daily exhibited 45% tumor growth inhibiton, while MRTX1133 dosed twice daily for 2 consecutive days exhibited 4% tumor growth inhibition at day 15 depending on dosing regimen. The combination of MRTX1133 and irinotecan resulted in a 83% tumor growth inhibition at day 15. See FIG. 1.

### Example C

KRas G12D inhibitor MRTX1133 in Combination with Irinorecan (PANC0203 Pancreatic Cancer Cell Line)

20 nude/nude mice were inoculated with Panc 02.03 cells in the hind right flank. When the tumors reached ˜300 mm³ four treatment groups were established with 5 mice per group. The results of this study are provided in Table 2:

TABLE 2
Average Tumor Volumes (mm³) of nude/nude Tumor Bearing
Mice Treated with Single Agents and in Combination

| | Vehicle | MRTX1133 | Irinotecan | Irino + |
|---|---|---|---|---|
| Study | BID | 30 mg/kg | 100 mg/kg | MRTX1133 |
| Day | Daily | BID 2x/week | Q7D | 2x/week |
| 1 | 328.896 | 329.18 | 327.15 | 327.354 |
| 4 | 446.116 | 265.246 | 369.406 | 299.040 |
| 8 | 544.424 | 386.354 | 372.346 | 312.220 |
| 11 | 647.874 | 392.042 | 402.394 | 296.390 |
| 15 | 786.634 | 480.434 | 408.086 | 283.703 |
| 18 | 927.746 | 496.496 | 392.356 | 278.697 |
| 22 | 1113.464 | 549.618 | 381.028 | 296.417 |

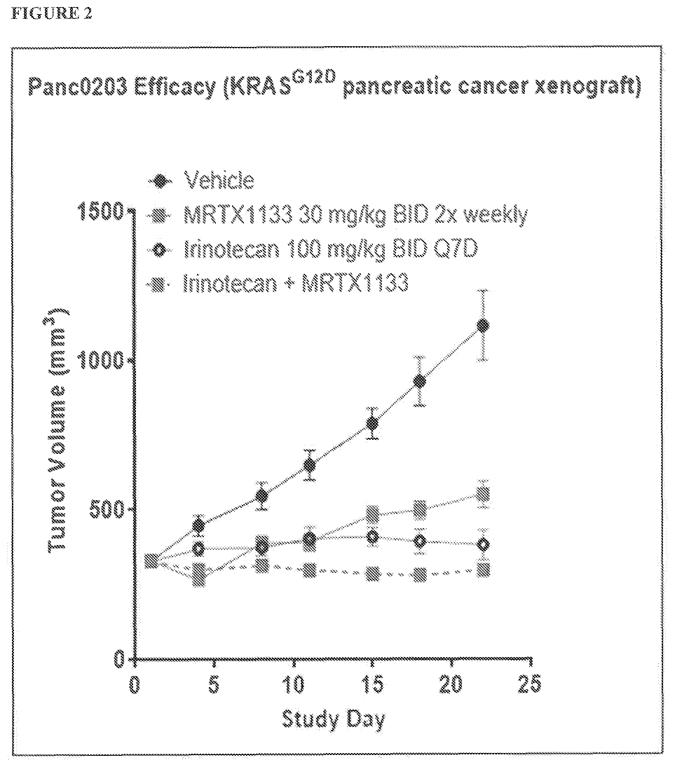

As shown in Table 2, the administration of MRTX1133 as a single agent exhibited 72% tumor growth at day 22. The combination of MRTX1133 and irinotecan resulted in a −9% tumor regression at day 22. See FIG. 2.

### Example D

KRas G12D inhibitor MRTX1133 in Combination with Irinorecan (SNU1033 Rectal Cancer Cell Line)

20 nude/nude mice were inoculated with SNU1033 cells in the right hind flank When tumors reached ˜˜300 mm³ four treatment groups were established with 5 mice per group. The results of this study are provided in Table 3:

TABLE 3
Average Tumor Volumes (mm³) of nude/nude Tumor Bearing
Mice Treated with Single Agents and in Combination

| | Vehicle | MRTX1133 | Irinotecan | Irino + |
|---|---|---|---|---|
| Study | BID | 30 mg/kg | 100 mg/kg | MRTX1133 |
| Day | Daily | 2x/week | Q7D | 2x/week |
| 1 | 294.992 | 298.92 | 306.118 | 310.004 |
| 5 | 487.128 | 336.336 | 458.222 | 356.884 |
| 8 | 560.436 | 431.99 | 454.256 | 365.158 |
| 12 | 727.846 | 468.892 | 476.136 | 348.536 |
| 15 | 826.37 | 529.222 | 526.648 | 319.35 |
| 19 | 1054.748 | 643.516 | 596.814 | 299.074 |
| 22 | 1225.776 | 703.368 | 630.514 | 279.864 |
| 26 | 1287.22 | 802.69 | 641.502 | 249.116 |
| 29 | 1350.962 | 838.708 | 632.96 | 205.048 |
| 33 | 1435.752 | 895.918 | 642.344 | 129.1 |

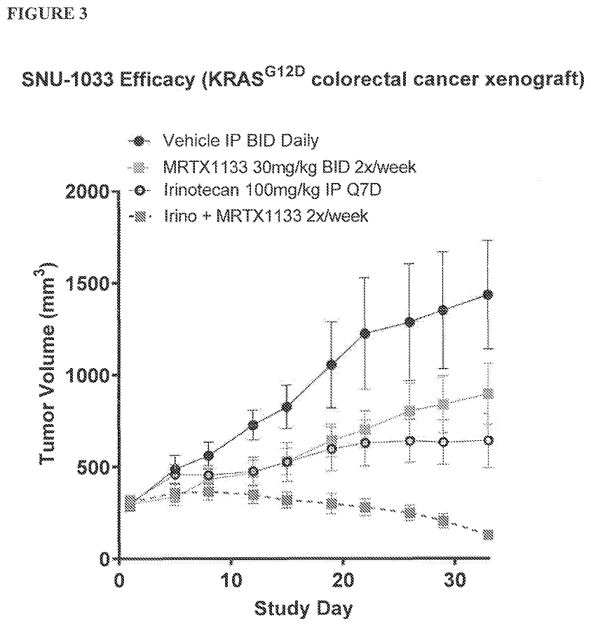

As shown in Table 3, the administration of MRTX1133 as a single agent exhibited 47% tumor growth inhibition at day 33. The combination of MRTX1133 and irinotecan resulted in a −58% tumor regression at day 33. See FIG. 3.

### Example E

KRas G12D Inhibitor MRTX1133 in Combination with Gemcitabine, and in Combination with Gemcitabine/nP (SNU1033, PA NC0203 and L8180 Cancer Cell Lines)

For each cell line (SNU1033, PANC0203 and LS180, and for each combination MRTX1133+gemcitabine, and MRTX1133+gemcitabine/nP): 20 nude/nude mice are inoculated with cells in the right hind flank When tumors reach ˜300 mm³ four treatment groups aree established with 5 mice per group. The results of this study are generated and provided in a table to generated.

These results demonstrate that the combination therapy resulted in greater amount of tumor growth inhibition compared to either single agent alone demonstrating enhanced in vivo anti-tumor efficacy of the combination against KRas G12D expressing cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of treating cancer in a subject in need thereof, comprising administering to the subject a therapeutically effective amount of a combination a KRas G12D inhibitor or a pharmaceutically acceptable salt thereof, and a cytotoxic compound selected from irinotecan, topotecan, belotecan, trastuzumab deruxtecan and camptothecin, or a pharmaceutically acceptable salt thereof.

2. The method of claim 1, wherein the KRas G12D inhibitor or salt is selected from: MRTX1133: 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynyl-6-fluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethynylnaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5,6-difluoronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-chloronaphthalen-2-ol; 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-ethyl-6-fluoronaphthalen-2-ol; 4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethylnaphthalen-2-ol; and 4-(4-((1R,5S)-3,8-diazabicyclo[3.2.1]octan-3-yl)-8-fluoro-2-(((2R,7aS)-2-fluorohexahydro-1H-pyrrolizin-7a-yl) methoxy)pyrido[4,3-d]pyrimidin-7-yl)-5-fluoronaphthalen-2-ol; and a pharmaceutically acceptable salt thereof, and where the cytotoxic compound is irinotecan.

3. The method of claim 1, wherein the KRas G12D inhibitor is MRTX1133: 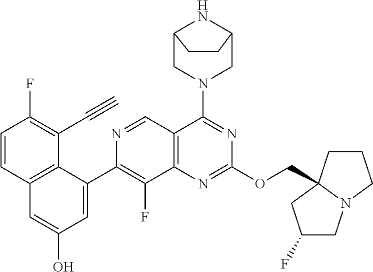 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, and the cytotoxic compond is irinotecan.

4. The method of according to claim 1, wherein the KRas G12D inhibitor or salt, and the cytotoxic compound, are administered on the same day.

5. The method of according to claim 1, wherein the KRas G12D inhibitor or salt, and the cytotoxic compound, are administered on different days.

6. The method according to claim 1, wherein the KRas G12D inhibitor or salt is administered at a maximum tolerated dose.

7. The method according to claim 1, wherein the cytotoxic compound is administered at a maximum tolerated dose.

8. The method according to claim 1, wherein the KRas G12D inhibitor or salt, and the cytotoxic compound, are each administered at a maximum tolerated dose.

9. The method according to claim 1, wherein the KRas G12D inhibitor or salt is administered at below maximum tolerated dose.

10. The method according to claim 1, wherein the cytotoxic compound is administered at below maximum tolerated dose.

11. The method according to claim 1, wherein the KRas G12D inhibitor or salt, and the cytotoxic compound, are each administered at below maximum tolerated dose.

12. The method according to claim 1, wherein the therapeutically effective amount of the combination of the KRas G12D inhibitor or salt and the cytotoxic compound results in an increased duration of overall survival, an increased duration of progression free survival, an increase in tumor growth regression, an increase in tumor growth inhibition or an increased duration of stable diseasein the subjects relative to treatment with only the KRas G12D inhibitor or salt.

13. The method according to claim 1, wherein the therapeutically effective amount of the combination of the KRas G12D inhibitor or salt, and the cytotoxic compound, results in an increased duration of overall survival, an increased duration of progression free survival, an increase in tumor growth regression, an increase in tumor growth inhibition or an increased duration of stable diseasein the subjects relative to treatment with only the cytotoxic compound.

14. A pharmaceutical composition comprising a therapeutically effective amount of a combination of a KRas G12D inhibitor or pharmaceutically acceptable salt thereof, and a cytotoxic compound selected from irinotecan, topotecan, belotecan, trastuzumab deruxtecan and camptothecin or a pharmaceutically acceptable salt thereof, and a pharmaceutically acceptable excipient.

15. The composition of claim 14, comprising MRTX1133: 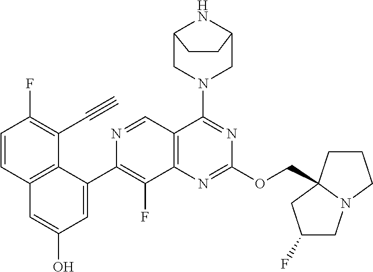 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, irinotecan, and a pharmaceutically acceptable excipient.

16. A method of method for inhibiting KRas G12D activity in a cell, comprising contacting the cell in which inhibition of KRas G12D activity is desired with an effective amount of a combination a KRas G12D inhibitor or a pharmaceutically acceptable salt thereof, and a cytotoxic compound selected from irinotecan, topotecan, belotecan, trastuzumab deruxtecan and camptothecin or a pharmaceutically acceptable salt thereof.

17. The method of claim 16, wherein the KRas G12D inhibitor is MRTX1133: 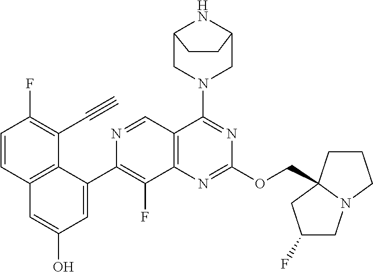 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
and pharmaceutically acceptable salts thereof, and the cytotoxic compound is irinotecan.

18. The method according to claim 1, wherein irinotecan increases the sensitivity of cancer cells to the KRas G12D inhibitor.

19. A method for increasing the sensitivity of a cancer cell to the KRas G12D inhibitor comprising administering to a subject undergoing KRas G12D treatment with an effective amount of a combination the KRas G12D inhibitor MRTX1133: 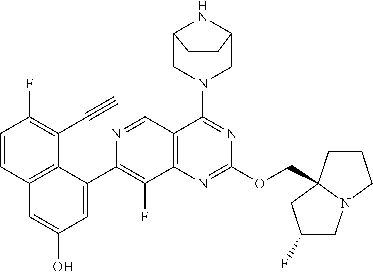 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]
or a pharmaceutically acceptable salt thereof, and irinotecan, wherein the irinotecan increases the sensitivity of the cancer cell to the KRas G12D inhibitor.

20. The method according to claim 1, wherein the therapeutically effective amount of the KRas G12D inhibitor in the combination is between about 0.01 to 100 mg/kg per day.

21. The method of claim 20, wherein the therapeutically effective amount of the KRas G12D inhibitor in the combination is between about 0.1 to 50 mg/kg per day.

22. The method according to claim 1, wherein the therapeutically effective amount of irinotecan in the combination is between about 0.01 to 100 mg/kg per day.

23. The method of claim 22, wherein the therapeutically effective amount of irinotecan in the combination is between about 0.1 to 50 mg/kg per day.

24. The method according to claim 1, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial′carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

25. The method of claim 23, wherein the cancer wherein the cancer is a KRas G12D-associated cancer.

26. The method of claim 23, wherein the cancer is pancreatic, colorectal, endrometrial and non-small cell lung cancer.

27. A kit comprising the pharmaceutical composition of claim 14 for treating KRas G12D cancer in a subject.

28. The kit according to claim 27, further comprising an insert with instructions for administration of the pharmaceutical composition.
